(19) United States
(12) Patent Application Publication
(10) Pub. No.: US 2002/0147406 A1
(43) Pub. Date: Oct. 10, 2002

(54) Cardiac stabilizers and methods of use thereof

(76) Inventor: Ludwig K. von Segesser, Lausanne (CH)

Correspondence Address: MINTZ, LEVIN, COHN, FERRIS,, GLOVSKY and POPEO, P.C., One Financial Center, Boston, MA, 02111, US

(21) Appl. No.: 10/114,199

(22) Filed: Apr. 2, 2002

Related U.S. Application Data
(60) Provisional application No. 60/280,817, filed on Apr. 2, 2001.

(51) Int. Cl. A61B 5/04; A61N 1/18

(52) U.S. Cl. 600/509; 600/37; 607/10

(57) ABSTRACT

The present invention relates to cardiac stabilizers and methods of use thereof in cardiac surgical procedures. In particular, the invention relates to the uses of cardiac stabilizers having one or more sensors operably attached to one or more stabilizing members to detect and treat reduced cardiac output during coronary bypass surgery.

### RELATED APPLICATIONS

This application claims the benefit of U.S. Ser. No. 60/280,817, filed Apr. 2, 2001, which is incorporated herein by reference in its entirety.

### FIELD OF THE INVENTION

The present invention relates generally to cardiac stabilizers useful in surgical procedures and to methods of use of such cardiac stabilizers. In particular, the invention relates to the uses of such stabilizers during coronary artery bypass surgery.

### BACKGROUND OF THE INVENTION

Diseases of the cardiovascular system affect millions of people each year and are a leading cause of death throughout the world. The cost to society from such diseases is enormous both in terms of the number of lives lost as well as in terms of the costs associated with treating patients through traditional surgical techniques. A particularly prevalent form of cardiovascular disease is a reduction in the blood supply leading to the heart caused by atherosclerosis or other conditions that create a restriction in blood flow at a critical point in the cardiovascular system that supplies blood to the heart.

Treatment of such a blockage or restriction in the blood flow leading to the heart is, in many cases, treated by a surgical procedure known as a coronary artery bypass (CAB) procedure, more commonly known as a “heart bypass” operation. In the CAB procedure, the surgeon “bypasses” the obstruction to restore normal blood flow to the heart either by attaching an available source vessel to the obstructed target coronary artery or by removing a portion of a vein or artery from another part of the body, to use as a graft, and installing the graft between a point on a source vessel and a point on a target artery.

In recent years, a growing number of surgeons have begun performing CAB procedures while the heart is still beating. Two such procedures are minimally invasive coronary artery bypass (MIDCAB) and off pump coronary artery bypass (OPCAB). During the anastomotic procedure between the bypass graft and the target coronary artery segment, the blood flow within this segment is reduced or interrupted, causing reduced blood flow to the myocardial tissue dependant on the coronary artery. Myocardial ischemia arrhythmia may result.

### SUMMARY OF THE INVENTION

In one aspect, the present invention involves an apparatus for detecting signals of a beating heart during a surgical procedure, this apparatus having a stabilizing member capable of contacting a local area of cardiac tissue in the beating heart at one or more contact areas, and a detection means for detecting the cardiac signals. In certain embodiments the contact between the apparatus and the cardiac tissue is accomplished using a contact-enhancing factor, e.g., mechanical force, suction created by a vacuum, one or more sutures, one or more conducting pins, a bio-compatible adhesive, or a combination of two or more of these factors. In certain other embodiments the apparatus additionally has a shaft with a proximal and a distal end, which, in some embodiments, may be substantially rigid. In other embodiments the shaft is malleable and can be made substantially rigid mechanically, chemically, or by human intervention; an example of this is a shaft composed of a flexible material surrounding a steel cable capable of being made rigid mechanically when the shaft is in an advantageous conformation.

In some embodiments, the detection means includes one or more sensors capable of measuring detectable physical, chemical, biochemical or biological signals, including but not limited to cardiac constriction signals, current, impedance, pH, pressure, oxygen saturation, temperature, ion elution, cardiac wall thickening, or a combination of two or more of these signals. In other embodiments, the detection means includes one or more electrocardiogram signal collecting means, e.g., one or more electrodes. The electrodes may be reversibly or irreversibly attached to the stabilizing member at one or more points along the member. In one embodiment, the apparatus may be used to detect cardiac constriction signals of a beating heart during a surgical procedure. In other embodiments, other sensors as would be known to those skilled in the art are used to detect cardiac signals during a surgical procedure.

In further embodiments, the apparatus contains two or more sensors arranged in a proximal to distal orientation relative to the trunk of the aorta along the local area or the cardiac tissue, so that the difference in one or more of detectable physical, chemical, biochemical or biological values between cardiac tissue proximal and distal to the contact area of the apparatus is determined. In another embodiment, the apparatus contains two or more sensors, arranged so that a first sensor is located opposite a second sensor relative to an incision within the cardiac tissue.

In another aspect, the present invention involves an apparatus for delivering an electrical stimulus to a local area of a heart during a surgical procedure, this apparatus having a stabilizing member capable of contacting a local area of cardiac tissue in the beating heart at one or more contact areas, and a stimulation means for delivering an electrical stimulus to the cardiac tissue. In some embodiments, the local area or cardiac tissue stimulated by the apparatus is located distally relative to the aorta from the site of the surgical procedure. In certain embodiments the contact between the apparatus and the cardiac tissue is accomplished using a contact-enhancing factor, e.g., mechanical force, suction created by a vacuum, one or more sutures, one or more conducting pins, a bio-compatible adhesive, or a combination of two or more of these factors. In certain other embodiments the apparatus additionally has a shaft, which may be substantially rigid. In other embodiments the shaft is malleable and can be made substantially rigid mechanically, chemically, or by human intervention.

In some embodiments, the stimulation means is one or more electrodes capable of delivering an electrical stimulus to the cardiac tissue. In other embodiments, the electrodes may be reversibly or irreversibly attached to the stabilizing member at one or more points along the member. The first electrode may be located distal to a second electrode relative to the trunk of the aorta. Alternatively, the first electrode may be located opposite a second electrode relative to an incision with the cardiac tissue.

In a further aspect, the present invention involves an apparatus for detecting one or more cardiac signals of a beating heart and delivering an electrical stimulus to a local area of the heart during a surgical procedure, this apparatus having a stabilizing member capable of contacting a local area of cardiac tissue in the heart at one or more contact areas between the apparatus and the beating heart; a detection means for detecting cardiac signals, and a stimulation means capable of delivering an electrical stimulus to the cardiac tissue.

In certain embodiments the contact between the apparatus and the cardiac tissue is accomplished using a contact-enhancing factor, e.g., mechanical force, suction created by a vacuum, one or more sutures, one or more conducting pins, a biocompatible adhesive, or a combination of two or more of these factors. In certain other embodiments the apparatus additionally has a shaft, which may be substantially rigid. In other embodiments the shaft is malleable and can be made substantially rigid mechanically, chemically, or by human intervention.

In some embodiments, the detection means includes one or more sensors capable of measuring detectable physical, chemical, biochemical or biological values, such as a cardiac constriction signal, current, impedance, pH, pressure, oxygen saturation, temperature, ion elution, cardiac wall thickening, or a combination of two or more of these values. In other embodiments, the detection means includes one or more electrocardiogram signal collecting means, e.g., one or more electrodes. The detection means and the stimulation means may be separate electrodes or they may be the same electrode.

In further embodiments, the apparatus contains two or more sensors arranged in a proximal to distal orientation relative to the aorta along the local area or the cardiac tissue, and a stimulation means, so that the difference in one or more of detectable physical, chemical, biochemical or biological values between cardiac tissue proximal and distal to the contact area of the apparatus is determined, and an electrical stimulation can be provided to the distal cardiac tissue if the values of the distal cardiac tissue are not equal to the values of the proximal cardiac tissue.

In a still further aspect, the present invention involves an apparatus for detecting one or more cardiac signals of a beating heart and delivering an electrical stimulus to a local area of the heart during a surgical procedure, this apparatus having a stabilizing member capable of contacting a local area of cardiac tissue in the heart at one or more contact areas between the apparatus and the beating heart and a shaft operably connected to the stabilizing member, whereby the shaft is capable of measuring pressure exerted by the heart. In an embodiment of the invention, the stabilizing member may itself be used as a detection means and/or a stimulation means. In some embodiments, the shaft is insulated from electrical conductance.

In yet another aspect, the present invention involves a method for monitoring the activity of a beating heart, by contacting the surface of a beating heart with an apparatus having a stabilizing member capable of contacting a local area of cardiac tissue in the beating heart at one or more contact areas, and a detection means for detecting the cardiac signals; and monitoring the activity of the beating heart. In one embodiment, this method of monitoring occurs during a CAB procedure. In other embodiments, the monitoring includes measuring a cardiac constriction signal, current, impedance, pH, pressure, oxygen saturation, temperature, ion elution, cardiac wall thickening, or a combination of two or more of these factors. In still other embodiments, the contact between the apparatus and the cardiac tissue is accomplished by a contact enhancing factor, e.g., mechanical force, suction created by a vacuum, one or more sutures, one or more conducting pins, a bio-compatible adhesive, or a combination of two or more of these factors. In an additional embodiment, the method further includes stimulating the contacted cardiac tissue, such as with an electrical stimulation. In a related embodiment, the method includes detecting one or more cardiac constriction signals of the contacted cardiac tissue and stimulating the contacted cardiac tissue in response to this monitored activity.

In another aspect, the present invention involves a method for preventing ischemic damage during coronary bypass surgery, by contacting the surface of a beating heart with an apparatus having a stabilizing member capable of contacting a local area of cardiac tissue in the heart at one or more contact areas between the apparatus and the beating heart, a detection means for detecting cardiac constriction signals, and a stimulation means capable of delivering an electrical stimulus to the cardiac tissue; performing the coronary bypass surgery on the local area of the beating heart; detecting one or more cardiac signals of the contacted cardiac tissue; and stimulating or pacing the contacted cardiac tissue, resulting in a predetermined heart rate in response to the cardiac constriction signals. Such stimulation prevents decreased cardiac output and/or reduces ischemic damage in the entire body, particularly the cardiac tissue distal to the trunk of the aorta relative to the contacted cardiac tissue.

In one embodiment, the detection means is capable of detecting one or more cardiac signals selected from the group consisting of cardiac constriction signals, current, impedance, pH, pressure, oxygen saturation, temperature, ion elution, cardiac wall thickening, or a combination thereof. Ischemic damage during coronary bypass surgery may be caused by irregular cardiac constriction signals during the surgical procedure. In various embodiments, the first cardiac constriction signal may be detected in a region distal to a second cardiac constriction signal relative to the aorta or in a region distal to an incision site in the cardiac tissue relative to the aorta. For example, the incision site is the site of an anastomosis in a coronary artery bypass surgery.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting.

Other features and advantages of the invention will be apparent from the following detailed description and from the claims.

### BRIEF DESCRIPTION OF THE DRAWINGS

FIG. 1 is a schematic illustration of an apparatus of the present invention. A stabilizing member 1 contains two substantially planar contact arms 4, one or more associated sensors 2, and may optionally contain a shaft 3. A sensor 2 may be a detection means, a stimulation means or both, and may have wire 5 leading from the sensor 2 through the shaft 3 and to an instrumentality capable of interpreting, storing, and/or displaying information provided by the sensor.

FIG. 2 illustrates the contact of an apparatus of the present invention with a heart during a surgical procedure, indicating regions proximal and distal to the site of the surgical procedure. A stabilizing member 1 contacts the heart 6 at one or more points in proximity to a coronary artery 7, which is located between planar contact arms 4. Shaft 3 is connected to a retractor 8. Sensors are shown at proximal 9 and distal 10 locations relative to the aorta 11.

### DETAILED DESCRIPTION TO THE INVENTION

In view of the potential myocardial ischemia and arrhythmia caused by reduction in blood flow during coronary artery bypass surgery, it would be desirable to have improved devices for monitoring the beating heart at the site of the anastomosis and correcting any procedure-dependent local myocardial ischemia and/or arrhythmia during the surgical procedure.

The present invention is directed in part to an apparatus and methods for locally stabilizing an anastomotic site of a beating heart during a cardiac surgical procedure such as OPCAB, while measuring signals produced by the beating heart and optionally delivering an electrical stimulation to a region of cardiac tissue in need thereof. While the present invention is described in detail as applied to coronary artery bypass surgical procedures performed on a beating heart, those skilled in the art will appreciate that the present invention can be applied to other surgical procedures and other internal organs where locally stabilizing tissue, measuring signals produced by the tissue, and optionally delivering an electrical stimulation to a region of the tissue in need thereof is a primary goal.

As used herein, the following definitions are supplied in order to facilitate the understanding of this case. To the extent that the definitions vary from meanings known to those skilled in the art, the definitions below control.

“Myocardial ischemia” is any acute or chronic condition caused or characterized by reduced blood flow to tissues of the heart.

“Arrhythmia” is any irregularity in the force or rhythm of the heartbeat.

“Anastomosis” is the union or connection of two or more blood vessels in the heart.

A “cardiac constriction signal” includes any electrical signal generated by the heart, e.g., an electrical signal measured by an electrocardiogram instrument (EKG or ECG).

“Cardiac tissue” includes all tissue of the heart, aorta and pericardium.

“Proximal” indicates that the location of an object is closer to a first reference point than a second object or second reference point. “Distal” indicates that the location of an object is farther away from a reference point than a second object or second reference point. As used herein, blood flows through the coronary arteries from proximal to distal points relative to the aortic trunk.

“Cardiac signal” as used herein includes, but is not limited to, cardiac constriction signal, current, impedance, pH, pressure, oxygen saturation, temperature, ion elution, and cardiac wall thickening.

“Contact-enhancing factor” as used herein includes, but is not limited to, mechanical force, suction created by a vacuum, one or more sutures, one or more conducting pins, and a bio-compatible adhesive.

“Pacing” as used herein refers to the providing of an electrical stimulation to the heart at one or more points to increase or stabilize the beating of the heart.

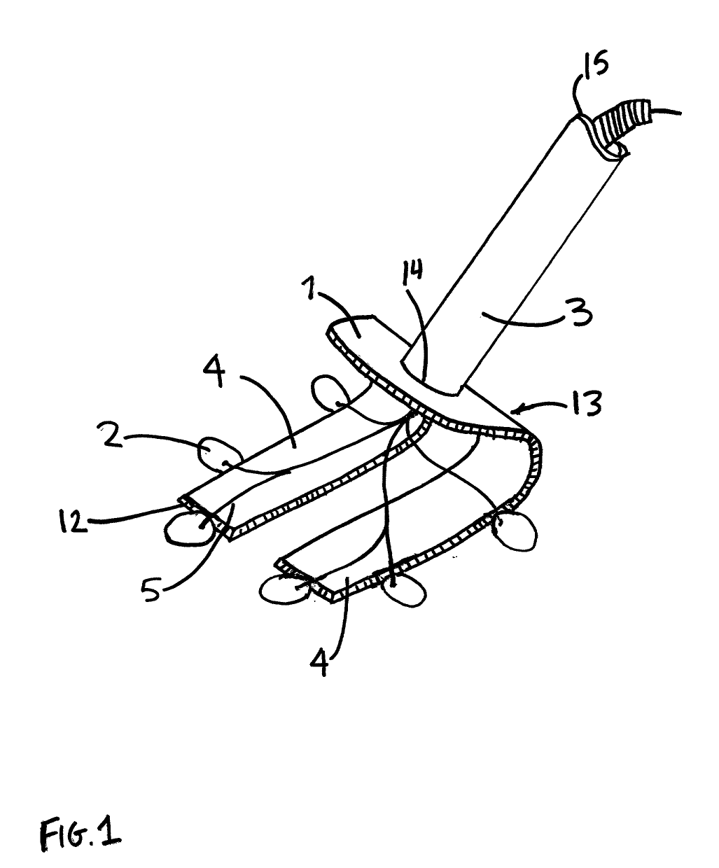

FIG. 1 shows a schematic representation of a preferred embodiment of the present invention. The apparatus contains a stabilizing member 1 having a distal end 12 and a proximal end 13, containing two substantially planar contact arms 4. The stabilizing member is operably linked to one or more associated sensors 2.

The present invention optionally provides a shaft 3 having a longitudinal axis and a distal end 14 and a proximal end 15, where the distal end of the shaft is operably linked to the proximal end of the stabilizing member. This shaft is useful as a handle for the apparatus and may be attached to a retractor or other fixed support structure (See, e.g., U.S. Pat. No. 6,290,644, which is incorporated herein by reference). The shaft may be substantially rigid, and may be hollow to accommodate wires leading from the one or more sensors operably linked to the stabilizing member to a device capable of receiving the signals detected by the one or more sensors. The wires leading from one or more sensors may be alternatively linked directly to a device capable of receiving the signals detected by the one or more sensors and not go through the shaft. Alternatively, the one or more sensors may communicate remotely (e.g., wirelessly or via a telemetry link) with a device capable of receiving the signals detected by the one or more sensors. Alternatively, the shaft may be malleable and capable of being made substantially rigid mechanically, chemically, or by human intervention, such as a shaft composed of a flexible material surrounding a steel cable capable of being made rigid mechanically when the shaft in an advantageous conformation. Additionally, the shaft may be adjustable in length and orientation relative to the stabilizing member.

While the apparatus of the present invention is described in detail as having a stabilizing member having two substantially planar contact arms, those skilled in the art will appreciate that the present invention can be practiced using any stabilizing member capable of immobilizing a local region of the heart while permitting the heart to beat as required in order to continue unabated cardiac output, to which one or more detection and/or stimulation means can be operably attached. The stabilizing member may be in the shape of a square, ring, bell, or any other useful geometry envisioned by one skilled in the art. (See, e.g., U.S. Pat. No. 6,361,493; U.S. Pat. No. 6,334,843; U.S. Pat. No. 6,019,722; U.S. Pat. No. 5,921,979; and U.S. Pat. No. 5,894,843, each of which is incorporated herein by reference).

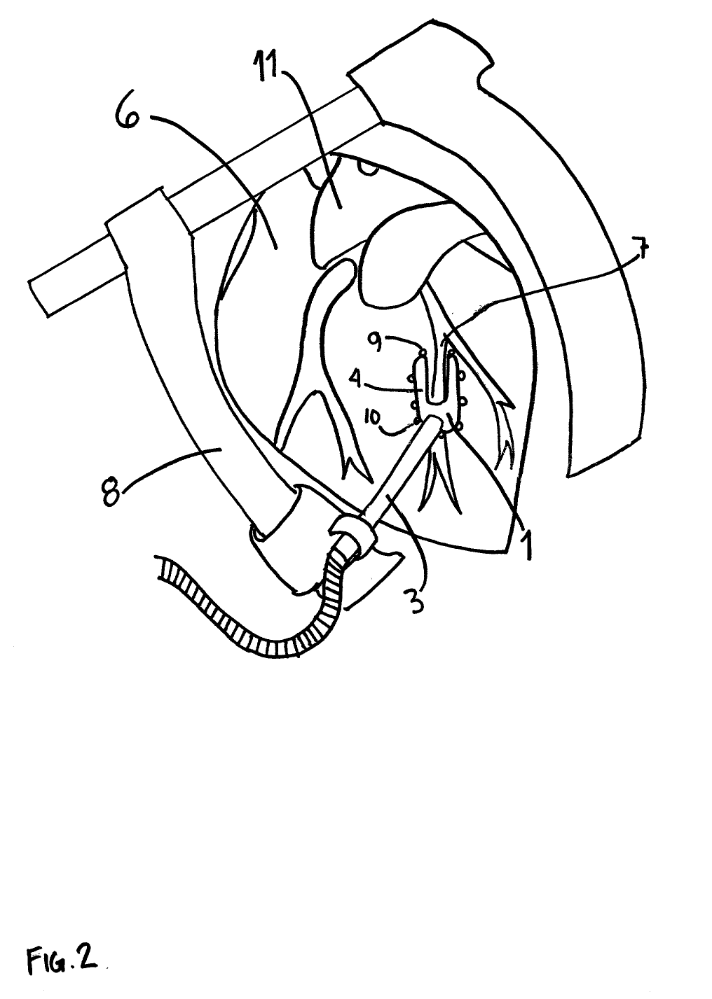

In one embodiment of the present invention, the stabilizing member has a pair of substantially planar contact arms 4 which contact each other at the proximal end 5 of the stabilizing member. Each of the planar contact arms 4 may be adjustable in length and orientation relative to the other contact arm, depending on the configuration of the tissue and the clinical aspects of the procedure performed. Each of the planar contact arms 4 may be curved or straight. Further, each of the planar contact arms 4 may be substantially rigid or malleable. Alternatively, the stabilizing member 1 may have more than or less than two contact arms. These contact arms engage the heart on either side of a target coronary artery as shown in FIG. 2. The cardiac tissue may also be contacted by one or more sensors such as a sensor located proximally 9 and a sensor located distally 10 relative to the aortic trunk.

The contact between the stabilizing member and the cardiac tissue may be facilitated by the use of a contact-enhancing factor, e.g., mechanical force, suction created by a vacuum, one or more sutures, one or more conducting pins, a bio-compatible adhesive, or a combination of two or more of these factors. One skilled in the art would be able to determine with minimal experimentation the contact-enhancing factor or combination of factors most useful in a particular clinical situation. Other contact-enhancing factors known to those skilled in the art are also within the scope of this invention.

The one or more sensors 2 operably linked to stabilizing arm 1 may include a detection means, a stimulation means or both. A detection means includes one or more sensors capable of measuring a cardiac signal, including but not limited to cardiac constriction signals, current, impedance, pH, pressure, oxygen saturation, temperature, ion elution, cardiac wall thickening, or a combination of two or more of these signals. Those skilled in the art will recognize that other signals may also be detected and/or measured. In preferred embodiments of the present invention, the sensors may be electrodes, such as electrodes capable of measuring cardiac constriction signals. Other detection means, including sensors that are known to those skilled in the art are also within the scope of this invention. Sensors that communicate remotely (e.g., wirelessly or via a telemetry link) with a device capable of receiving the signals detected by the sensors are also encompassed by this invention. Suitable sensors for use as part of the present invention include, for example thosedescribed in U.S. Pat. No. 6,314,323; U.S. Pat. No. 6,295,466; U.S. Pat. 6,309,350; U.S. Pat. No. 6,312,380; U.S. Pat. 6,289,238; U.S. Pat. No. 6,299,583; U.S. Pat. No. 5,601,084; and U.S. Pat. No. 5,908,028, which are incorporated by reference in their entireties.

The detection means (e.g., the sensors that measure cardiac signals) can be oriented along the stabilizing member such that they are useful in measuring one or more cardiac signals, as described above. The first sensor may be located in a region distal to a second sensor relative to the aorta, or in a region distal to an incision site in the cardiac tissue relative to the aorta. Alternatively, the first sensor may be located in a region proximal to a second sensor relative to the aorta, or in a region proximal to an incision site in the cardiac tissue relative to the aorta. For example, the incision site is the site of an anastomosis in a coronary artery bypass surgery. Such signals are indicative of the viability of the cardiac tissue distal to the site of the anastomosis and will alert the surgeon that local myocardial ischemia is developing in the distal regions of the heart. Thus, the occurrence and regression of procedure-dependent local ischemia can be monitored and counteracted by modifying the surgical techniques and/or pharmacological intervention, or electrical stimulation of the ischemic cardiac tissue.

The one or more sensors may also be a stimulation means, e.g., an electrode, which is capable of delivering an electrical stimulus to the cardiac tissue. The detection means and the stimulation means may be the same one or more electrodes or they may be different one or more electrodes. Thus, the occurrence of procedure-dependent local ischemia, which results in decreased cardiac output and/or ischemic damage to the entire body, can be monitored, and if necessary, counteracted by providing an electrical stimulation to the affected cardiac tissue. Such stimulation will pace the contacted cardiac tissue, thereby restoring a predetermined heart rate such that proper cardiac output is restored and ischemic damage to the entire body is reduced or prevented.

The present invention also provides an apparatus for detecting one or more cardiac signals of a beating heart and delivering an electrical stimulus to a local area of the heart during a surgical procedure in response to the signals. This apparatus has a stabilizing member capable of contacting a local area of cardiac tissue in the heart at one or more contact areas between the apparatus and the beating heart and a shaft operably connected to the stabilizing member, whereby the stabilizing member and shaft are capable of measuring one or more cardiac signals (e.g., measuring the pressure exerted by the heart by monitoring the forces exerts on the stabilizing member or shaft). The stabilizing member may itself be used as a detection means and/or a stimulation means, provided that the stabilizing member and shaft are insulated or physically separated from any object (such as a retractor or spreader) which may inhibit or reduce the conductance of the signal.

The present invention further provides a method for monitoring the activity of a beating heart, by contacting the surface of a beating heart with an apparatus having a stabilizing member 1 capable of contacting a local area of cardiac tissue in the beating heart at one or more contact areas, and a detection means for detecting the cardiac signals, such as a sensor located proximally 9 and a sensor located distally 10 relative to the aortic trunk; and monitoring the activity of the beating heart with these sensors. This method of monitoring cardiac activity is useful during a CAB procedure, where the surgical procedure causes procedure-dependent local ischemia in the cardiac tissue near the site of surgery, which can result in decreased cardiac output which can, in turn, produce ischemic damage to the entire body. The monitoring may include measuring a cardiac constriction signal, current, impedance, pH, pressure, oxygen saturation, temperature, ion elution, cardiac wall thickening, or a combination of two or more of these factors. The contact between the apparatus and the cardiac tissue is accomplished by a contact enhancing factor, e.g., mechanical force, suction created by a vacuum, one or more sutures, one or more conducting pins, a bio-compatible adhesive, or a combination of two or more of these factors. Additionally, the cardiac tissue may be stimulated, such as with electrical stimulation, in response to the monitored activity. Providing such electrical stimulation to the cardiac tissue may pace the contacted cardiac tissue at a predetermined heart rate, thereby restoring cardiac output and preventing or reducing ischemic effects over the entire body.

The invention also provides a method for preventing ischemic damage during coronary bypass surgery, by contacting the surface of a beating heart with an apparatus having a stabilizing member 1 capable of contacting a local area of cardiac tissue in the heart 6 at one or more contact areas between the apparatus and the beating heart, a detection means for detecting cardiac constriction signals, and a stimulation means capable of delivering an electrical stimulus to the cardiac tissue; performing the coronary bypass surgery on the local area of the beating heart; detecting one or more cardiac signals of the contacted cardiac tissue; and stimulating (“pacing”) the contacted cardiac tissue, resulting in a predetermined heart rate in response to the cardiac constriction signals. Such stimulation may prevent decreased cardiac output and reduce ischemic damage in the entire body, particularly the cardiac tissue distal to the trunk of the aorta relative to the contacted cardiac tissue. Additionally, the detection means may also be capable of detecting one or more cardiac signals selected from the group consisting of cardiac constriction signals, current, impedance, pH, pressure, oxygen saturation, temperature, ion elution, cardiac wall thickening, or a combination thereof, or other cardiac signals known to those skilled in the art.

The first cardiac constriction signal may be detected in a region distal to a second cardiac constriction signal relative to the aorta, or in a region distal to an incision site in the cardiac tissue relative to the aorta. For example, the incision site is the site of an anastomosis in a coronary artery bypass surgery. Alternatively, the first cardiac constriction signal may be detected in a region proximal to a second cardiac constriction signal relative to the aorta, or in a region proximal to an incision site in the cardiac tissue relative to the aorta. For example, the incision site is the site of an anastomosis in a coronary artery bypass surgery.

### OTHER EMBODIMENTS

It is to be understood that, while the invention has been described in conjunction with the detailed description thereof, the foregoing description is intended to illustrate and not limit the scope of the invention, which is defined by the scope of the appended claims. Other aspects, advantages, and modifications are within the scope of the following claims.

I claim:

1. An apparatus for detecting signals of a beating heart during a surgical procedure, the apparatus comprising:
i) a stabilizing member capable of contacting a local area of cardiac tissue in said beating heart at one or more contact areas between said apparatus and said beating heart, and
ii) a detection means for detecting the cardiac signals.

2. The apparatus of claim 1, wherein said detection means comprises one or more sensors capable of measuring a cardiac constriction signal, current, impedance, pH, pressure, oxygen saturation, temperature, ion elution, cardiac wall thickening, or a combination of two or more of these factors.

3. The apparatus of claim 1, wherein said detection means comprises one or more electrocardiogram signal collecting means.

4. The apparatus of claim 3, wherein said detection means comprises two or more electrocardiogram signal collecting means.

5. The apparatus of claim 4, wherein a first electrocardiogram signal collecting means is located distal to a second electrocardiogram signal collecting means relative to the trunk of the aorta.

6. The apparatus of claim 4, wherein a first electrocardiogram signal collecting means is located proximal to a second electrocardiogram signal collecting means relative to the trunk of the aorta.

7. The apparatus of claim 2, wherein a first sensor is located distal to a second sensor relative to the trunk of the aorta.

8. The apparatus of claim 2, wherein a first sensor is located proximal to a second sensor relative to the trunk of the aorta.

9. The apparatus of claim 4, wherein a first electrocardiogram signal collecting means is located opposite a second electrocardiogram signal collecting means relative to an incision within the cardiac tissue.

10. The apparatus of claim 1, wherein said contact between said apparatus and said cardiac tissue is accomplished by a contact enhancing factor selected from the group consisting of mechanical force, suction created by a vacuum, one or more sutures, one or more conducting pins, a bio-compatible adhesive, or a combination of two or more of these factors.

11. The apparatus of claim 1, further comprising a shaft having a proximal and a distal end, said stabilizing member being operably connected to said distal end.

12. The apparatus of claim 10, wherein said shaft is substantially rigid.

13. The apparatus of claim 11, wherein said shaft is malleable and can be made rigid.

14. The apparatus of claim 3, wherein said electrocardiogram signal collecting means is one or more electrodes.

15. The apparatus of claim 14, wherein said one or more electrodes are reversibly attached to said stabilizing member at one or more points on said member.

16. The apparatus of claim 14, wherein said one or more electrodes are irreversibly attached to said stabilizing member at one or more points on said member.

17. An apparatus for detecting cardiac constriction signals of a beating heart during a surgical procedure, the apparatus comprising:
i) a stabilizing member capable of contacting a local area of cardiac tissue in said beating heart at one or more contact areas between said apparatus and said beating heart, and
ii) a detection means for detecting the cardiac constriction signals, wherein said detection means comprises one or more electrodes.

18. An apparatus for delivering an electrical stimulus to a local area of a heart during a surgical procedure, the apparatus comprising:
i) a stabilizing member capable of contacting a local area of cardiac tissue in the heart at one or more contact areas between said apparatus and said heart, and
ii) a stimulation means capable of delivering an electrical stimulus to said cardiac tissue.

19. The apparatus of claim 18, wherein said stimulation means comprises one or more electrodes.

20. The apparatus of claim 19, wherein said stimulation means comprises two or more electrodes.

21. The apparatus of claim 20, wherein a first electrode is located distal to a second electrode relative to the trunk of the aorta.

22. The apparatus of claim 20, wherein a first electrode is located proximal to a second electrode relative to the trunk of the aorta.

23. The apparatus of claim 20, wherein a first electrode is located opposite a second electrode relative to an incision within the cardiac tissue.

24. The apparatus of claim 18, wherein said contact between said apparatus and said cardiac tissue is accomplished by a contact enhancing factor selected from the group consisting of mechanical force, suction created by a vacuum, one or more sutures, one or more conducting pins, a bio-compatible adhesive, or a combination of two or more of these factors.

25. The apparatus of claim 18, further comprising a shaft having a proximal and a distal end, said stabilizing member being operably connected to said distal end.

26. The apparatus of claim 25, wherein said shaft is substantially rigid.

27. The apparatus of claim 25, wherein said shaft is malleable and can be made rigid.

28. An apparatus for detecting one or more cardiac signals of a beating heart and delivering an electrical stimulus to a local area of the heart during a surgical procedure, comprising:
i) a stabilizing member capable of contacting a local area of cardiac tissue in the heart at one or more contact areas between said apparatus and said beating heart;
ii) a detection means for detecting cardiac signals; and
iii) a stimulation means capable of delivering an electrical stimulus to said cardiac tissue.

29. The apparatus of claim 28, wherein said detection means comprises one or more sensors capable of measuring a cardiac constriction signal, current, impedance, pH, pressure, oxygen saturation, temperature, ion elution, cardiac wall thickening, or a combination of tow or more of these signals.

30. The apparatus of claim 28, wherein said detection means comprises at least one electrocardiogram signal collecting means comprising one or more first electrodes, and wherein said stimulation means comprises one or more second electrodes.

31. The apparatus of claim 29, wherein said detection means and said stimulation means comprise the same electrode.

32. The apparatus of claim 28, further comprising a shaft having a proximal and a distal end, said stabilizing member being operably connected to said distal end.

33. The apparatus of claim 32, wherein said shaft is substantially rigid.

34. The apparatus of claim 32, wherein said shaft is malleable and can be made rigid.

35. The apparatus of claim 28, wherein said contact between said apparatus and said cardiac tissue is accomplished by a contact enhancing factor selected from the group consisting of mechanical force, suction created by a vacuum, one or more sutures, one or more conducting pins, a bio-compatible adhesive, or a combination of two or more of these factors.

36. An apparatus for detecting one or more cardiac signals of a beating heart during a surgical procedure, comprising:
i) a stabilizing member capable of contacting a local area of cardiac tissue in the heart at one or more contact areas between said apparatus and said beating heart; and
ii) a shaft operably connected to said stabilizing member,
whereby said shaft is capable of detecting a cardiac signal.

37. An apparatus for detecting one or more cardiac signals of a beating heart and delivering an electrical stimulus to a local area of the heart during a surgical procedure, comprising:
i) a stabilizing member capable of contacting a local area of cardiac tissue in the heart at one or more contact areas between said apparatus and said beating heart; and
ii) a shaft operably connected to said stabilizing member,
whereby said stabilizing member operably connected to said shaft is capable of delivering an electrical stimulus to said heart.

38. The apparatus of claim 37, wherein said shaft is insulated from electrical conductance.

39. A method of monitoring the activity of a beating heart comprising:
a) contacting the surface of a beating heart with the apparatus of claim 26 at one or more contact areas; and
b) monitoring the activity of the beating heart.

40. The method of claim 39, wherein said monitoring occurs during coronary artery bypass surgery.

41. The method of claim 39, wherein said monitoring comprises measuring a cardiac constriction signal, current, impedance, pH, pressure, oxygen saturation, temperature, ion elution, cardiac wall thickening, or a combination of two or more of these factors.

42. The method of claim 39, wherein said contact between said apparatus and said beating heart is accomplished by a contact enhancing factor selected from the group consisting of mechanical force, suction created by a vacuum, one or more sutures, one or more conducting pins, a bio-compatible adhesive, or a combination of two or more of these factors.

43. The method of claim 39, further comprising detecting one or more cardiac constriction signals of said contacted cardiac tissue.

44. The method of claim 39, further comprising stimulating said contacted cardiac tissue.

45. The method of claim 39, further comprising detecting one or more cardiac constriction signals of said contacted cardiac tissue and stimulating said contacted cardiac tissue in response to said monitored activity.

46. A method of preventing ischemic damage during coronary bypass surgery, comprising:
a) contacting the surface of a beating heart with the apparatus of claim 28;
b) performing the coronary bypass surgery on the local area of the beating heart;
c) detecting one or more cardiac signals of said contacted cardiac tissue; and
d) stimulating said contacted cardiac tissue in response to said cardiac constriction signals;
wherein said stimulation prevents deterioration of cardiac output or reduces ischemic damage in the entire body.

47. The method of claim 46, wherein said stimulation prevents deterioration of cardiac output or reduces ischemic damage in cardiac tissue distal to the trunk of the aorta relative to the contacted cardiac tissue.

48. The method of claim 46, wherein said detecting of one or more cardiac signals comprises measuring a cardiac constriction signal, current, impedance, pH, pressure, oxygen saturation, temperature, ion elution, cardiac wall thickening, or a combination of two or more of these signals.

49. The method of claim 46, wherein said ischemic damage is caused by irregular cardiac constriction signals to cardiac tissue during coronary bypass surgery.

50. The method of claim 46, wherein a first cardiac constriction signal is detected in a region distal to a second cardiac constriction signal relative to the aorta.

51. The method of claim 46, wherein a first cardiac constriction signal is detected in a region proximal to a second cardiac constriction signal relative to the aorta.

52. The method of claim 46, wherein a first cardiac constriction signal is detected in a region distal to an incision site in the cardiac tissue relative to the aorta.

53. The method of claim 46, wherein a first cardiac constriction signal is detected in a region proximal to an incision site in the cardiac tissue relative to the aorta.

54. The method of claim 53, wherein said incision site is a site of anastomosis in a coronary artery bypass surgery.
